(19) United States
(12) Patent Application Publication
(10) Pub. No.: US 2004/0162514 A1
(43) Pub. Date: Aug. 19, 2004

(54) System and method for controlling differential pressure in a cardio-vascular system

(75) Inventors: Clifton Anton Alferness, Redmond, WA (US); John Mathew Adams, Sammamish, WA (US); David Kaye, Beaumaris (AU)

Correspondence Address: MERCHANT & GOULD PC, P.O. BOX 2903, MINNEAPOLIS, MN, 55402-0903, US

(73) Assignee: Scout Medical Technologies, Kirkland, WA

(21) Appl. No.: 10/367,671

(22) Filed: Feb. 14, 2003

(51) Int. Cl. A61M 5/00

(52) U.S. Cl. 604/9

(57) ABSTRACT

Disclosed are a system, device, and method for controlling a differential fluid pressure between chambers in a heart. The device includes an implantable pop-off valve that may be secured between two chambers of a heart, such as the left and right atriums. The pop-off valve is normally in a closed position. If a differential fluid pressure across the device exceeds a pre-determined value, the pop-off valve is arranged to open to enable blood flow between the two chambers of the heart. When it is determined that the differential fluid pressure is substantially below the pre-determined value, the pop-off valve is configured to close. The system and method further discloses means for implanting the device into a septal region of the heart.

### FIELD OF THE INVENTION

The present invention relates to controlling a cardio-vascular differential pressure and in particular to an implantable septal pop-off device and method for controlling a differential blood pressure between corresponding chambers in a heart.

### BACKGROUND OF THE INVENTION

Heart disease is the leading cause of death in the United States, with approximately 500,000 deaths each year resulting from heart attacks. A heart attack is commonly preceded by a history of hypertension, which is a sustained elevation of the systemic blood pressure (typically 160/95 mmHg or higher). Hypertension, the medical term for high blood pressure, is known as “the silent killer.” Close to 50 million Americans have hypertension, and as many as 17 million don't even know they have the condition. If left untreated, hypertension greatly increases the risk of heart attack and stroke, and quite often leads to death.

Hypertension can cause both diastolic and systolic heart failure. In diastolic heart failure, the heart works to pump against the increased arterial pressure, and the heart muscle thickens to compensate for the greater amount of work. This thickening of the heart muscle, called hypertrophy, can temporarily help the heart pump against the increased pressure. Eventually, however, the hypertrophied heart muscle becomes stiff and inflexible. Unable to properly fill itself with blood, the heart stops beating properly, resulting in diastolic heart failure.

In systolic heart failure, the pressure inside the left ventricle increases as the heart pumps. After some time, the ventricle walls begin to weaken, causing it to expand and stretch the heart out of shape. This damaging process is called dilation, and it severely impairs the heart's ability to pump forcefully. The result is systolic heart failure.

Moreover, during congestive heart failure, acute episodes of heart failure may arise due to increased circulation of blood volume. The increased blood volume results in the pressure in the left atrium (or ventricle) being substantially higher than the pressure in the right atrium (or ventricle) creating a deleterious differential pressure across the left and right atriums (or ventricles). If left untreated, the pressure may extend the left ventricular and atrium, potentially damaging and weakening the heart. Therefore, it would be beneficial to limit the cardio-vascular differential pressure. It is with respect to these considerations and others that the present invention has been made.

### SUMMARY OF THE INVENTION

This summary of the invention section is intended to introduce the reader to aspects of the invention. Particular aspects of the invention are pointed out in other sections herein below, and the invention is set forth in the appended claims, which alone demarcate its scope.

The present invention is directed to an apparatus, system, and method for controlling a differential fluid pressure in a cardio-vascular system. In one aspect of the invention, the apparatus includes a pressure sensitive valve that is configured to remain closed until a fluid pressure gradient across the valve reaches a first pre-determined value. Once the fluid pressure gradient reaches the pre-determined value, the valve pops open to enable fluid to flow through the valve. When the pressure gradient falls substantially below the pre-determined value, the valve is further configured to close.

In one aspect of the invention, the valve is directed to controlling a differential fluid pressure between corresponding left and right chambers of a heart. The valve includes a support base and a substantially rigid member. The support base includes a surface that is adapted for positioning over a passageway in tissue that is disposed between a right chamber and a corresponding left chamber. The member extends substantially parallel to the passageway. The member includes a fixed end and a free end. The fixed end of the member is attached towards a perimeter of the support base. The free end of the member is oriented towards a center of the support base. A higher fluid pressure in the left chamber causes a flap at an end of the passageway to open and flex the free end of the member away from the center of the support base, which enables blood to flow from the left chamber to the right chamber. A reduction in the differential fluid pressure enables a flexure of the free end of the member to close the flap at the end of the passageway and stop the flow of blood between the heart chambers.

In another aspect of the invention, a system is directed to controlling a differential fluid pressure between corresponding left and right chambers of a heart with a valve and a catheter means. The valve includes a support base and a substantially rigid member. The support base includes a surface that is adapted for positioning over a passageway in tissue that is disposed between a right chamber and a corresponding left chamber. The member extends substantially parallel to the passageway. The member includes a fixed end and a free end. The fixed end of the member is attached towards a perimeter of the support base. The free end of the member is oriented towards a center of the support base. A higher fluid pressure in the left chamber causes the passageway to open and flex the free end of the member away from the center of the support base. A reduction in the higher fluid pressure enables a flexure of the free end of the member to close the passageway. The catheter means enables the positioning of the valve over the passageway in the septum.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description of the Invention, which is to be read in association with the accompanying drawings, wherein:

FIG. 1A is a schematic cross section of a heart illustrating one embodiment of a pop-off valve deployed through a membranous septum;

FIG. 1B is a schematic cross section of a heart illustrating one embodiment of a pop-off valve deployed through the interatrial septum;

FIG. 2 is a cross-sectional view of a membrane showing the placement of one embodiment of the pop-off valve with anchor guides;

FIG. 3 is a cross-sectional view of a membrane showing the placement of another embodiment of the pop-off valve with anchor guides;

FIG. 4 is a schematic diagram illustrating two embodiments of pop-off valves that employ anchor guides;

FIG. 5 is a cross-sectional view showing one embodiment of a pop-off valve fully retracted within a catheter;

FIG. 6 is a cross-sectional view illustrating one embodiment of another pop-off valve partially retracted into a catheter; and

FIG. 7 is a cross-sectional view showing the pop-off valve partially retracted into one embodiment of a catheter that employs slicing blades, in accordance with the present invention.

### DETAILED DESCRIPTION OF THE PREFERRED EMBODIMENT

In the following detailed description of exemplary embodiments of the invention, reference is made to the accompanied drawings, which form a part hereof, and which is shown by way of illustration, specific exemplary embodiments of which the invention may be practiced. Each embodiment is described in sufficient detail to enable those skilled in the art to practice the invention, and it is to be understood that other embodiments may be utilized, and other changes may be made, without departing from the spirit or scope of the present invention. The following detailed description is, therefore, not to be taken in a limiting sense, and the scope of the present invention is defined only by the appended claims.

Throughout the specification and claims, the following terms take the meanings explicitly associated herein, unless the context clearly dictates otherwise.

The terms “comprising,” “including,” “containing,” “having,” and “characterized by,” mean an open-ended or inclusive transitional construct and does not exclude additional, unrecited elements, or method steps. For example, a combination that comprises A and B elements, also reads on a combination of A, B, and C elements.

The meaning of “a,” “an,” and “the” include plural references. The meaning of “in” includes “in” and “on.” Additionally, a reference to the singular includes a reference to the plural unless otherwise stated or is inconsistent with the disclosure herein.

Briefly stated, the present invention is directed to controlling a differential fluid pressure between a corresponding left and right chamber of a heart. In one embodiment of the present invention, a passageway for blood flow is formed by an incision through tissue between the two chambers. The passageway includes a flap of tissue at one end. If the flap is closed, blood does not flow through the passageway and when the flap is open, blood is free to flow between the two chambers. In one embodiment, the passageway is formed in a foramen ovale between the right and left auricles. In another embodiment, the passageway is formed in a membranous septum between the right and left ventricular chambers of the heart. In still another embodiment, the passageway is formed in a fossa ovalis. A valve of the present invention is secured over the flap of the passageway. The valve includes substantially rigid members extending substantially parallel to the flap. In one embodiment, the members comprise a biocompatible, shape memory material that enables the members to flex at a free end. When a pre-determined differential fluid pressure arises in the left chamber, the flap opens and the free end of each member flexes (or pops) away from the passageway, which enables fluid, such as blood, to flow from the left chamber into the right chamber. A drop in the differential fluid pressure substantially below the pre-determined fluid pressure enables a flexure in the free end of each of the members to close the flap and stop blood flow through the passageway.

For simplicity, the present invention will be described below primarily in the context of a left atrial pressure management device. However, the device and methods herein are readily applicable to a wider variety of pressure management procedures, and all such applications are contemplated by the present invention. For example, a left ventricle pressure management device is contemplated, wherein the device is secured to a septum region and over a passageway between the left and right ventricles.

Illustrative Environment

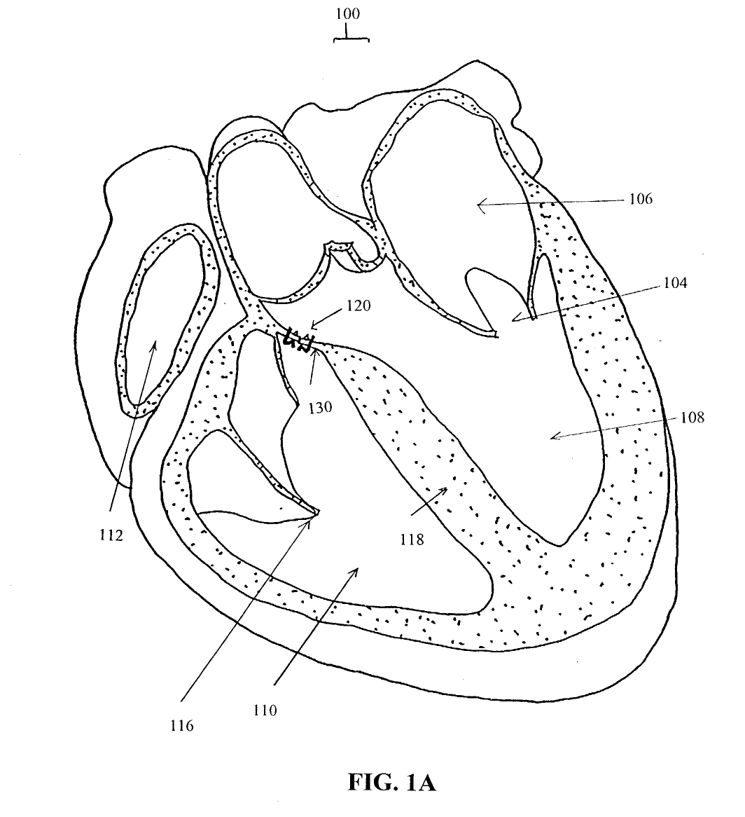

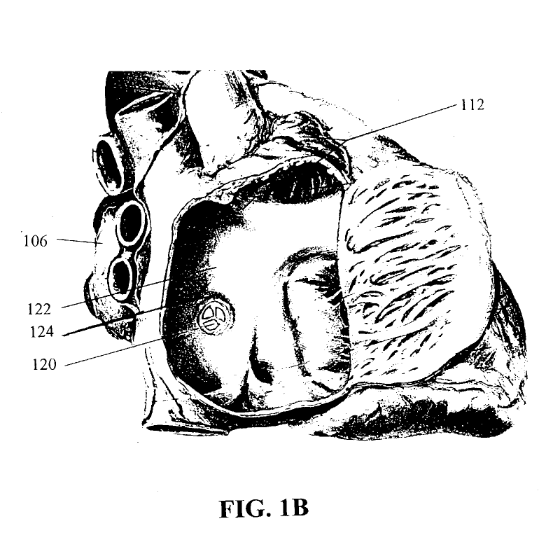

FIG. 1A is schematic cross section through heart 100 with one embodiment of a pop-off valve deployed through an interatrial septum. As shown in the cross sectional figure, heart 100 includes left atrium 106, left ventricle 108, right ventricle 110, right atrium 112, aorta 102, mitral valve 104, tricuspid valve 116, interventricular septum 118, and membranous septum 130. Also shown in the figure is pop-off valve 120.

As is understood in the art, left atrium 106, is located above left ventricle 108 and the two are separated by mitral valve 104. Left atrium 106 is normally in fluid communication with left ventricle 108 such that blood flows in and out of left ventricle 108 as heart 100 beats. Similarly, right atrium 112 is located above right ventricle 110 and the two are separated by tricuspid valve 116. Right atrium 112 is normally in fluid communication with right ventricle 110 such that blood flows in and out of right ventricle 110 as heart 100 beats.

Moreover, left ventricle 108 and right ventricle 110 are normally separated by interventricular septum 118. Similarly, left atrium 106 and right atrium 112 may be separated by interatrial septum 122 as shown in FIG. 1B. Also shown in FIG. 1B is fossa ovalis 124.

During the prenatal stage of development, the heart is typically created from the fusion of two ventral aortas that form a single pulsating organ. Separation into right and left heart chambers takes place later with a formation and closing of interatrial septum 122 and interventricular septum 118 or membranous septum 130. Before birth, the blood is typically oxygenated in a placenta and returned to right atrium 112 through an inferior vena cava (not shown). The blood is then directed by a Eustachian valve (not shown) through the foramen ovale, which is a persistent opening in interatrial septum 122. After birth, the foramen ovale may close, but the opening in interatrial septum 122 may persist to a varying degree in an adult. In one embodiment, the present invention employs the foramen ovale in establishing a passageway through interatrial septum 122 for fluid flow. For example, pop-off valve 120 can be positioned and secured over the passageway created by the foramen ovale to control the fluid flow from left atrium 106 to right atrium 112. Pop-off value 120 may also be positioned and secured through fossa ovalis 124 in interatrial septum 122 as illustrated in FIG. 1B, such as when the foramen ovale is closed.

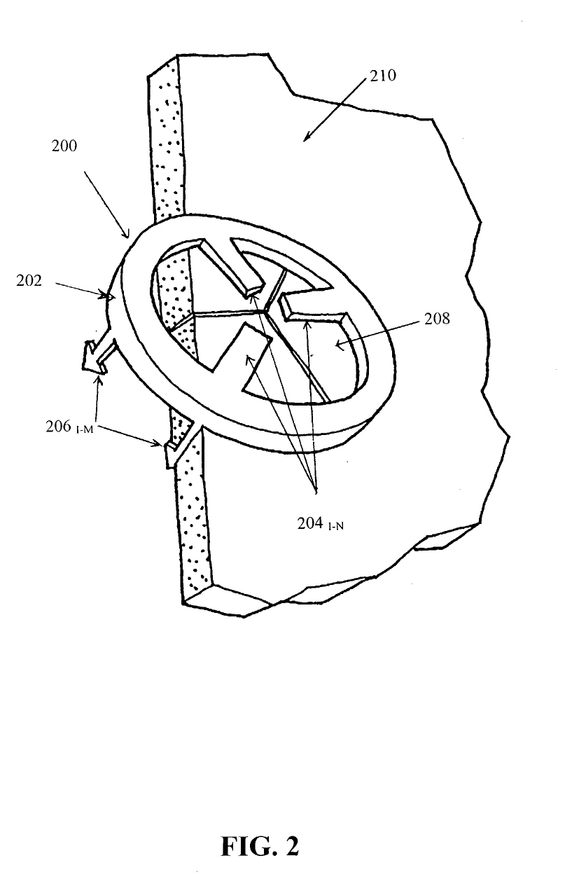

FIG. 2 is a cross-sectional view of a membrane showing a placement of one embodiment of the pop-off valve with anchor guides. As shown, device 200 includes support base 202, members 204_{1-N}, and anchor guides 206_{1-M}.

Also shown in the figure are flaps 208, which are located at an end of a passageway through tissue 210, which is disposed between left and right chambers of the heart (not shown). Support base 202 includes a surface that is adapted for positioning over flaps 208 in the passageway. As shown, support base 202 is substantially annular in shape. However, in other embodiments, support base 202 may be oval, rectangular, or virtually any other shape that enables members 204_{1-N} to be affixed to it, without departing from the scope or spirit of the present invention.

Support base 202 further includes anchor guides 206_{1-M}, each of which are spaced approximately equidistance around the perimeter of support base 202 and at approximately 90 degrees to the fixed ends of members 204_{1-N}. Anchor guides 206_{1-M} are employed to secure device 200 over flaps 208 for a passageway disposed between the left and right chambers of the heart.

The number of anchor guides, M, may range from one to virtually any number that is configured to minimize injury, while securing support base 202 to tissue 210. However, device 200 is not constrained to just employing anchor guides. Rather, device 200 may be secured to a membrane of the heart by a variety of other mechanisms, including, but not limited to, sewing, clamping, pinning, gluing, screwing, and the like, without departing from the scope or spirit of the present invention.

As shown in FIG. 2, members 204_{1-N} each has a fixed end and a free end. The fixed ends are attached approximately equidistance around the perimeter of support base 202. The free ends of members 204_{1-N} are oriented towards a center of support base 202.

Members 204_{1-N} are configured to allow each free end to flex open or pop away from pressure exerted by flaps 208 that is caused by a pre-determined differential fluid pressure between the two chambers of the heart. Members 204_{1-N} are further configured to enable a flexure at the free end to close underlying flaps 208 in response to a reduction in the differential fluid pressure between the two chambers of the heart.

In one embodiment of the present invention, when device 200 is positioned over a passageway through interatrial septum 122 shown in FIG. 1A, members 204_{1-N} are configured to flex open in response to a differential fluid pressure that exceeds approximately 10 to approximately 20 mmHg between left and right atriums (106 and 112). In another embodiment, when device 200 is positioned over a passageway through interventricular septum 118 shown in FIG. 1A, members 204_{1-N} are configured to flex open in response to a differential fluid pressure that exceeds approximately 140 mmHg to approximately 170 mmHg between left and right ventricles (108 and 110).

Although, FIG. 2 illustrates the number of members 204_{1-N}, as three (N), the present invention is not so constrained. The number of members, N, may be one, two, three, or more, arranged such that at least one member extends substantially parallel to at least one flap at one end of a passageway through tissue disposed between left and right chambers of a heart.

Members 204_{1-N} may include an elemental composition of a shape memory alloy (SMA) material. SMA material is configured to exhibit a stress-induced phase change to achieve high levels of elastic, reversible strain without permanent deformation to store and release significant levels of energy. The SMA material may extend longitudinally along members 204_{1-N}. In one embodiment, members 204_{1-N}, support base 202, and anchor guides 206_{1-M} are comprised of a single unitary SMA material.

The SMA material may include alloys of at least some of the following elements: Nickel, Gold, Silver, Cadmium, Indium, Gallium, Manganese, Cobalt, Carbon, Nitrogen, Silicon, Germanium, Tin, Zinc, Niobium, Copper, Iron, Platinum, Thallium, Aluminum, Chromium, Antimony, Carbon, and Titanium. A commercially available, useful SMA is NITINOL®, which is a Titanium-Nickel alloy. More generally, useful SMAs exhibit stress-induced, reversible, austenitic-martenistic phase transformations to exhibit superelastic (reversible strain) properties without permanent deformation at useful strain levels. Dimensional sizing and composition of the SMA members would preferably allow reversible strain levels without permanent, plastic deformation and allow the members to store and release greater energy than would be possible without SMA over multiple flexures of members 204_{1-N}.

Members 204_{1-N} may also include any other synthetic material having superelastic, biocompatible properties. For example, members 204_{1-N} may include a polymer, such as polyurethane, or similar synthetic material. Members 204_{1-N} may further include springy stainless steel, or titanium without departing from the scope or spirit of the present invention.

Moreover, support base 202, and anchor guides 206_{1-M} may also include an elemental composition of a shape memory alloy (SMA) material, or a synthetic material having superelastic, biocompatible properties. Support base 202, and anchor guides 206_{1-M} may further include other materials including, but not limited to stainless steel, titanium, and the like. Additionally, device 200 may be coated with an anticoagulant such as heparin, warfarin, low molecular weight heparin, Ticlopidine, Clopidgreland, Clopidogrel, and the like.

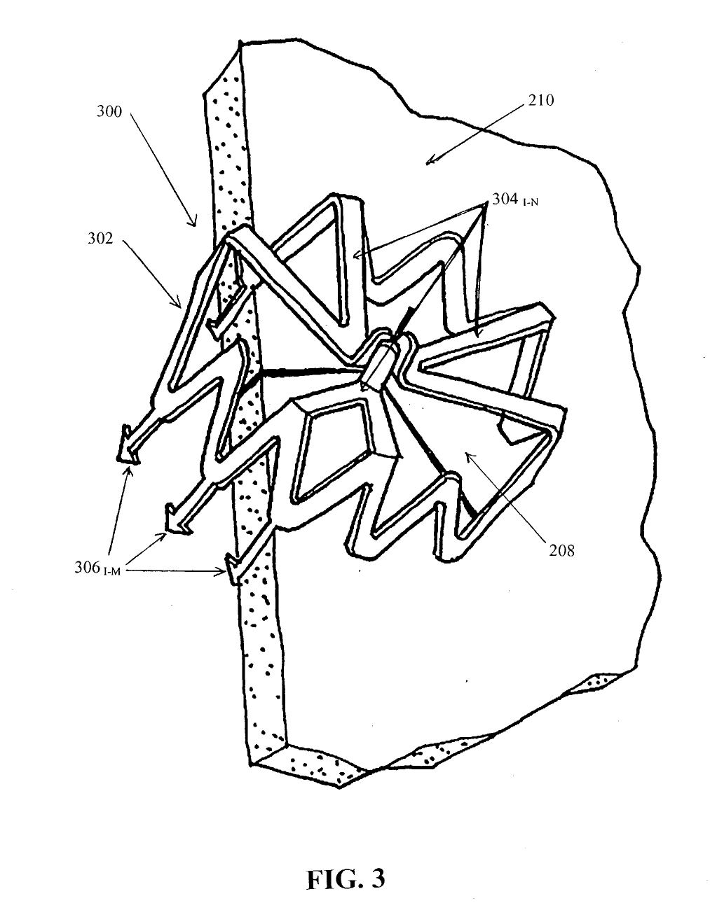

Briefly, FIG. 3 is a cross-sectional view of a membrane in the heart showing the placement of another embodiment of the pop-off valve device with anchor guides. As illustrated in FIG. 3, device 300 includes support base 302, members 304_{1-N}, and anchor guides 306_{1-M}, in a substantially similar configuration as device 200 shown in FIG. 2. Moreover, each of the components of device 300 is substantially similar in function, composition, and the like, of each of the respective components of device 200.

However, as shown in FIG. 3, each member 304 is approximately triangular shaped, with two fixed ends. Approximately the middle of each member 304 is operable as a free end, that functions in a manner substantially similar to member 204 in FIG. 2. Members 304_{1-N} are configured to enable a wider dispersion of support to underlying flap 208 than members 204_{1-N} under certain conditions.

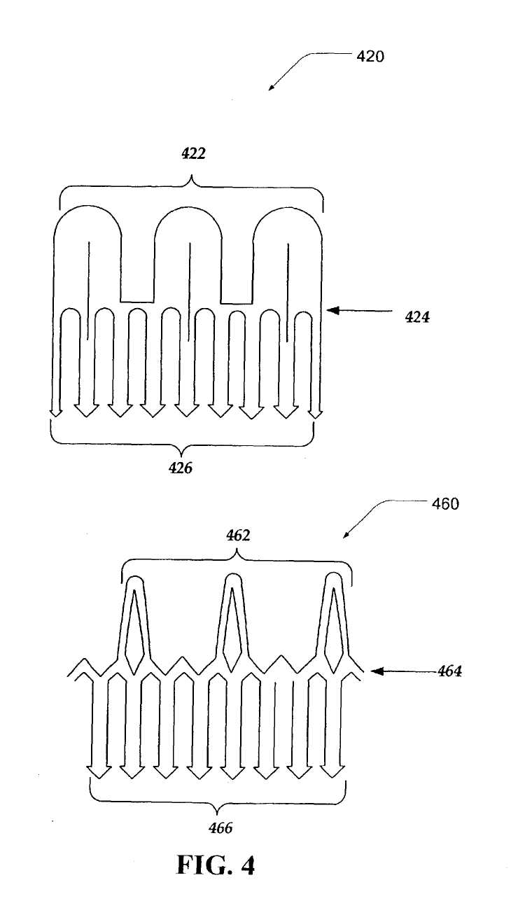

FIG. 4 is a schematic diagram illustrating two embodiments for constructing pop-off valve devices employing anchor guides. Form 420 includes members 422, base support 424, and anchor guides 426 as a unitary construct. Form 460 includes members 462, base support 464, and anchor guides 466 as another unitary construct.

As shown in FIG. 4, device 200 may be constructed employing form 420. Similarly, device 300 may be constructed employing form 460. In addition, either form 420 or form 460 may be constructed from a single sheet of material that is configured and shaped into device 200 and device 300, respectively.

Generalized Operation

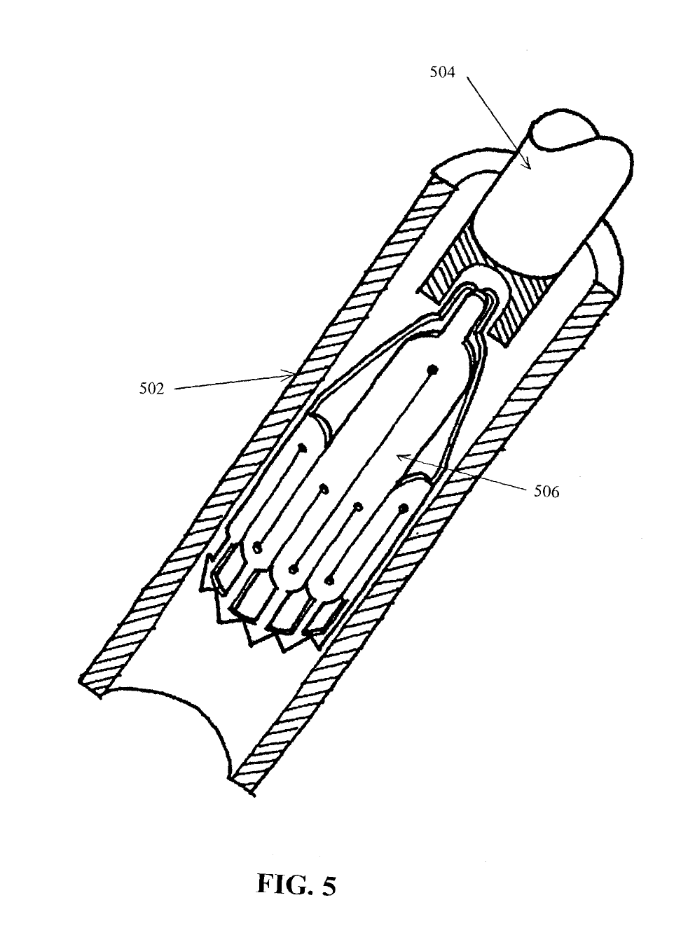

Implantation of a pop-off valve device in accordance with one method of the present invention is now described with respect to FIGS. 5-7. In accordance with one embodiment of the present invention, a catheter is advanced through the heart and into right atrium 112 or right ventricle 110. The catheter is adapted to position a pop-off valve device through a heart membrane, such as interatrial septum 120, interventricular septum 118, or the like. The right atrium 112 (or right ventricle 110) may be accessed through any of a variety of vessel pathways. For example, transeptal access may be achieved by introducing a transeptal catheter through a femoral or jugular vein, and transluminally advancing the catheter into right atrium 112. Once in right atrium 112, a radiopaque contrast media may be injected to allow visualization and ensure placement of the pop-off valve in tissue disposed between corresponding left and right chambers of a heart, as opposed to being in a pericardial space, aorta, or other undesirable location. The catheter may also include a piezoelectric ultrasound element for mapping the anatomy of the heart.

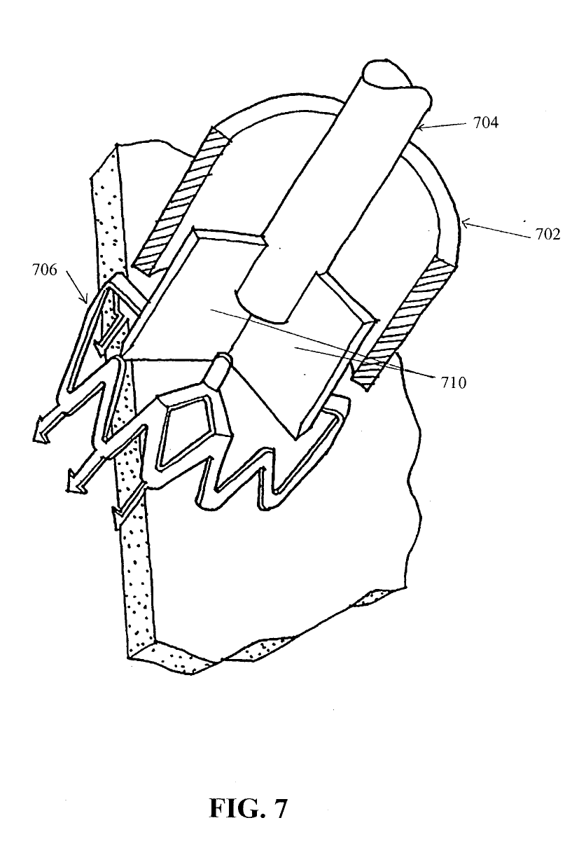

If required, at least one incision may be made into the tissue disposed between the two corresponding left and right heart chambers to establish a passageway for enabling fluid flow between the two chambers. In one embodiment, the passageway is formed in the foramen ovale through interatrial septum 122. Various surgical instruments be employed, including but not limited to, a medical laser, a mechanical cutting element, such as a rotating blade, or a cannulating needle. Briefly referring to FIG. 7, catheter 700 illustrates slicing blades 710 for forming an incision in tissue.

Additional incisions may be made in a manner forming at least one flap over the surgically prepared passageway. However, the flaps may be artificial, tissue, or a combination of artificial material and tissue. For example, the flaps may be formed from a polymer such as polyurethane, springy stainless steel, or similar synthetic material that is substantially inert in the human body.

The catheter is positioned such that a retracted pop-off valve device may be inserted over the passageway. Briefly referring to FIG. 5 a cross-sectional view of one embodiment shows a pop-off valve device fully retracted into catheter 500.

As shown in FIG. 5, pusher device 504 is advanced within catheter sheath 502 to urge retracted pop-off valve device 506 out of catheter sheath 502 and into the septal membrane surrounding the passageway.

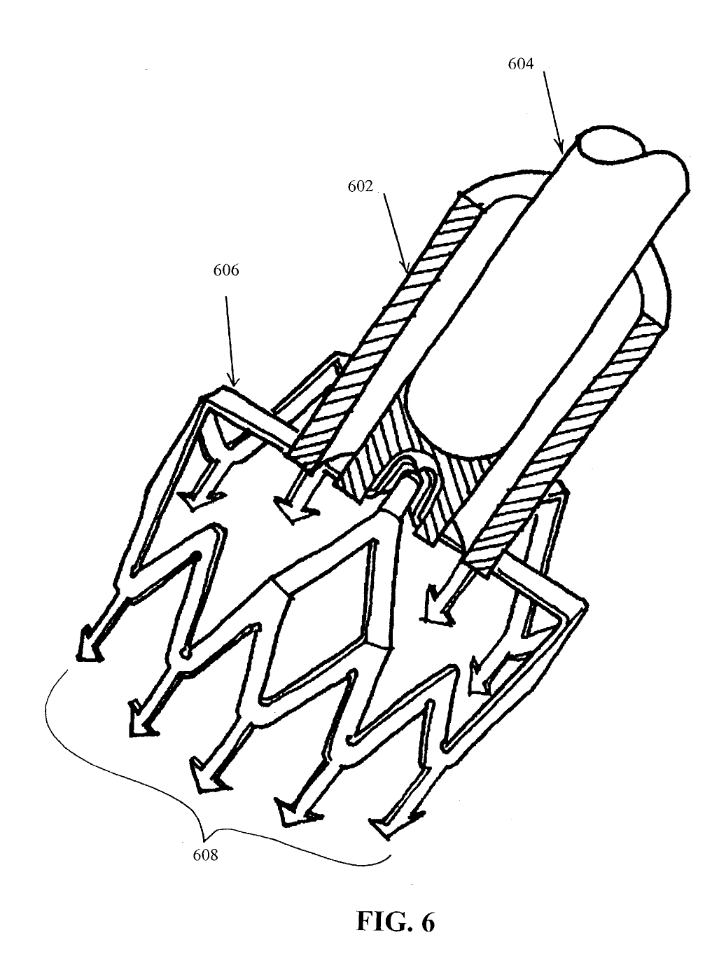

FIG. 6 shows a cross-sectional view of one embodiment of another pop-off valve device partially retracted for implantation into the septal membrane. As shown, partially retracted pop-off valve device 606 includes anchor guides 608 arranged to penetrate and secure device 606 to the membrane of the septum. Securing a pop-off valve device to the heart septum may also be achieved by a variety of other mechanisms. For example, a pop-off valve device may be secured to the surrounding septal membrane by sewing, clamping, pinning, gluing, screwing, and the like.

The pop-off valve is secured such that each of its members extends substantially parallel to at least one flap at an end of the passageway. When properly implanted in a heart, the pop-off valve will control fluid pressure between the two heart chambers substantially as described above.

The implantation of the pop-off valve device need not be performed with a catheter. Instead, surgical procedures, thoracoscopic procedures, and the like may be employed without departing from the scope or spirit of the present invention.

The above specification, examples, and data provide a complete description of the manufacture and use of the composition of the invention. Since many embodiments of the invention can be made without departing from the spirit and scope of the invention, the invention resides in the claims hereinafter appended.

We claim:

1. An apparatus for controlling a differential fluid pressure between a left chamber and a right chamber of a heart, comprising:
(a) a support base with a surface that is adapted for positioning over a passageway disposed between the right chamber and the left chamber; and
(b) a substantially rigid member that extends substantially parallel to the passageway, wherein a fixed end of the member is attached to the support base and a free end of the member is oriented away from the support base, and wherein a higher differential fluid pressure in the left chamber causes the passageway to open and flex the free end of the member towards the support base until a reduction in the differential fluid pressure enables a flexure of the free end of the member to close the passageway.

2. The apparatus of claim 1, wherein the support base is substantially annular in shape.

3. The apparatus of claim 1, further comprising a biocompatible synthetic material that forms at least one of the support base and the member.

4. The apparatus of claim 1, further comprising a shape memory material that forms at least one of the support base and the member.

5. The apparatus of claim 4, wherein the shape memory material further comprises a material that consists of at least one of a polymer, stainless steel, Nickel, Gold, Silver, Cadmium, Indium, Gallium, Manganese, Cobalt, Carbon, Nitrogen, Silicon, Germanium, Tin, Zinc, Niobium, Copper, Iron, Platinum, Thallium, Aluminum, Chromium, Antimony, Carbon, and a Titanium-Nickel alloy.

6. The apparatus of claim 1, further comprising an anti-clotting material that coats at least one of the support base and the member.

7. The apparatus of claim 1, wherein the passageway is formed in tissue located in at least one of an interventricular septum, an interatrial septum, a membranous septum, and a fossa ovalis.

8. The apparatus of claim 1, wherein the base support further comprises a means for securing the device over the passageway.

9. The apparatus of claim 1, wherein the higher differential fluid pressure exceeds about 10 to about 20 mmHg if the passageway is disposed in an interatrial septal region.

10. The apparatus of claim 1, wherein the higher fluid pressure exceeds about 140 to about 170 mmHg if the passageway is located in at least one of an interventricular septal region, and a membranous septum.

11. The apparatus of claim 1, further comprising a flap that is formed at an end of the passageway, wherein the free end of the member is disposed over and substantially parallel to the flap.

12. A system for controlling a differential fluid pressure between a left chamber and a right chamber of a heart, comprising:
(a) a valve, comprising:
(i) a support base with a surface that is adapted for positioning over a passageway disposed between the right chamber and the left chamber; and
(ii) a substantially rigid member that extends substantially parallel to the passageway, wherein a fixed end of the member is attached to the support base and a free end of the member is oriented away from the support base, and wherein a higher differential fluid pressure in the left chamber opens the passageway and flexes the free end of the member towards the support base until a reduction in the differential fluid pressure enables a flexure of the free end of the member to close the passageway; and
(b) a catheter means for positioning the valve over the passageway.

13. The system of claim 12, wherein at least one of the support base and the member further comprises a biocompatible synthetic material.

14. The system of claim 12, wherein at least one of the support base and the member further comprises a shape memory material.

15. The system of claim 12, wherein at least one of the support base and the member is coated with an anti-clotting material.

16. The system of claim 12, wherein the passageway is formed in one of an interventricular septum, an interatrial septum, a membranous septum, and a fossa ovalis.

17. The system of claim 12, wherein the support base is secured over the passageway by at least one of sewing, clamping, pinning, gluing, and screwing.

18. The system of claim 12, wherein the catheter further comprises a means for creating the passageway between the left chamber and the right chamber.

19. The system of claim 12, wherein the catheter further comprises a pusher for urging the valve out of the catheter.

20. The system of claim 12, further comprising a flap that is formed at an end of the passageway, wherein the free end of the member is disposed over and substantially parallel to the flap.

21. A method of controlling a differential fluid pressure between a left chamber and a right chamber of a heart, comprising:
(a) preparing a passageway between the left chamber and the right chamber, wherein the passageway enables fluid to flow between the left chamber and the right chamber; and
(b) securing a valve over the passageway, wherein the valve includes:
(a) a support base with a surface that is adapted for positioning over the passageway; and
(b) a substantially rigid member that extends substantially parallel to the passageway, wherein a fixed end of the member is attached towards a perimeter of the support base and a free end of the member is oriented away from the support base, and wherein a higher differential fluid pressure in the left chamber causes the passageway to open and flex the free end of the member towards the support base until a reduction in the differential fluid pressure enables a flexure of the free end of the member to close the passageway.

22. A valve for controlling a differential fluid pressure between a left atrium and a right atrium of a heart, comprising:
(a) a support base with a surface that is adapted for securing over a passageway in an interatrial septal region that is disposed between the right atrium and a corresponding left atrium;
(b) a substantially rigid member that extends substantially parallel to the passageway, wherein a fixed end of the member is attached towards a perimeter of the support base and a free end of the member is oriented away from the perimeter of the support base, and wherein a higher differential fluid pressure in the left atrium causes the passageway to open and flex the free end of the member towards the perimeter of the support base until a reduction in the differential fluid pressure enables a flexure of the free end of the member to close the passageway.

23. The valve of claim 22, wherein the support base is substantially annular in shape.

24. The valve of claim 22, wherein the substantially rigid member further comprises a shape memory material.

25. The valve of claim 22, wherein the valve is coated with an anti-clotting material.

26. The valve of claim 22, wherein the passageway is formed in tissue that comprises a foramen ovale.

27. The valve of claim 22, further comprising a flap that is formed at an end of the passageway, wherein the free end of the member is disposed over and substantially parallel to the flap.

28. A valve for controlling a differential blood pressure between a left ventricle and a right ventricle of a heart, comprising:
(a) a support base with a surface that is adapted for securing over a passageway in an interventricular septal region;
(b) a substantially rigid member that extends substantially parallel to the passageway, wherein a fixed end of the member is attached to the support base and a free end of the member is oriented towards a center of the support base, and wherein a higher differential blood pressure in the left ventricle causes the passageway to open and flex the free end of the member away from the center of the support base until a reduction in the differential blood pressure enables a flexure of the free end of the member to close the passageway.

29. A valve for enabling a passageway to open between a left chamber and a right chamber of a heart if a differential fluid pressure between the left chamber and the right chamber exceeds a pre-determined value.
